SEQUENCE LISTING (1 sequence)

SEQ ID NO: 1 - protein, 71 aa, Anabaena flos-aquae
MAVEKTNSSSSLAEVIDRILDKGIVVDAWVRVSLVGIELLAIEARIVIASVETYLKYAEAVGLTQSAAMPA

(19) United States
(12) Patent Application Publication
(10) Pub. No.: US 2014/0288411 A1
(43) Pub. Date: Sep. 25, 2014

(54) GAS VESICLE MAGNETIC RESONANCE IMAGING CONTRAST AGENTS AND METHODS OF USING THE SAME

(71) Applicant: The Regents of the University of California, Oakland, CA (US)

(72) Inventors: Mikhail Georgievich Shapiro, San Francisco, CA (US); Richard Matthew Ramirez, Wichita Falls, TX (US); Vikram Bajaj, El Cerrito, CA (US); Lindsay Joslyn Sperling, Dublin, CA (US); David Vernon Schaffer, Danville, CA (US); Alexander Pines, Berkeley, CA (US)

(21) Appl. No.: 14/206,974

(22) Filed: Mar. 12, 2014

Related U.S. Application Data
(60) Provisional application No. 61/778,106, filed on Mar. 12, 2013.

(51) Int. Cl. A61K 49/18

(52) U.S. Cl. CPC A61K 49/18
USPC 600/420; 530/300; 530/391.1; 424/9.37; 428/402.2

(57) ABSTRACT

Magnetic resonance imaging contrast agents that include a plurality of gas vesicles configured to associate with a noble gas are provided. Also provided are magnetic resonance imaging methods that include administering to a subject a contrast agent that includes a plurality of gas vesicles, obtaining a magnetic resonance data of a target site of interest, and analyzing the data to produce a magnetic resonance image of the target site. The subject contrast agents and methods find use in magnetic resonance imaging applications.

### CROSS-REFERENCE TO RELATED APPLICATIONS

Pursuant to 35 U.S.C. §119 (e), this application claims priority to the filing date of the U.S. Provisional Patent Application Ser. No. 61/778,106 filed Mar. 12, 2013; the disclosure of which application is herein incorporated by reference.

### REFERENCE TO GOVERNMENT SUPPORT

This invention was made with government support under grant number R01 ES020903 awarded by the National Institutes of Health. The Government has certain rights in this invention.

### INTRODUCTION

Magnetic Resonance Imaging (MRI) is a medical imaging technique used in radiology to visualize internal structures of the body. MRI makes use of the property of nuclear magnetic resonance (NMR) to image nuclei of atoms inside the body. An MRI scanner is a device in which a subject is positioned within a large, powerful magnet where the magnetic field is used to align the magnetization of some atomic nuclei in the subject, and radio frequency magnetic fields are applied to systematically alter the alignment of this magnetization. This causes the nuclei to produce a rotating magnetic field detectable by the scanner, and this information is recorded to construct an image of the scanned area of the body. Many natural and synthetic biological processes tied to gene expression occur in intact organisms or opaque specimens, contexts in which MRI provides monitoring capabilities. However, MRI lacks sensitive genetic reporters analogous to the green fluorescent protein (GFP) used in optical applications.

Previous attempts to develop molecular reporters for MRI have suffered from the low molecular sensitivity of the reporters. All such reporters developed so far rely on signal changes produced via their effect on thermally polarized ¹H nuclei, and thus the reporters are required to be present in concentrations sufficient to interact with a substantial fraction of ˜100 molar ¹H, primarily of water molecules, on sub-second timescales. As a result, practical detection limits have been in the micromolar range, compared to nanomolar for GFP.

### SUMMARY

Magnetic resonance imaging contrast agents that include a plurality of gas vesicles configured to associate with a noble gas are provided. Also provided are magnetic resonance imaging methods that include administering to a subject a contrast agent that includes a plurality of gas vesicles, obtaining a magnetic resonance data of a target site of interest, and analyzing the data to produce a magnetic resonance image of the target site. The subject contrast agents and methods find use in magnetic resonance imaging applications.

### BRIEF DESCRIPTION OF THE FIGURES

FIG. 1A shows a diagram of ¹²⁹Xe chemical exchange saturation transfer between bulk aqueous solvent (left) and GVs (hexagons) either in isolation or inside a cell (gray), according to embodiments of the present disclosure. Polarized ¹²⁹Xe nuclei (black) exchange into GVs, where they have a unique NMR frequency (red) at which they can be saturated by RF pulses. Saturated (gray) xenon returns to the bulk, causing a decrease in bulk ¹²⁹Xe signal. FIG. 1B shows NMR spectra of ¹²⁹Xe in buffer containing 400 pM GVs after saturation for the specified amount of time at 31.2 ppm. Spectra are offset for visibility. FIG. 1C shows a frequency-dependent saturation spectra for intact (red) and collapsed (black) GVs. Each spectrum is an average of two. FIG. 1D shows transmission electronmicrographs of intact (left, center) and collapsed (right) GVs. Scale bars are 200 nm. FIG. 1E shows a graph of concentration dependence of saturation contrast generated by GVs with saturation times corresponding color-wise to FIG. 1B. N=3 per data point. Data are fitted with monoexponential curves as a visual aide. FIG. 1F shows a saturation contrast image of a three-compartment phantom containing 400 pM GVs, 100 pM GVs and buffer. RF saturation and image averaging parameters are listed in Tables 1 and 2.

FIG. 2 shows a graph and images indicating that gas vesicles in different species of bacteria have distinct hyper-CEST saturation frequencies enabling multiplexed imaging, according to embodiments of the present disclosure. FIG. 2A shows a graph of frequency-dependent saturation spectra for solutions of wild-type Halobacteria NRC-1 (OD600=0.01), Microcystis sp. (OD600=0.36) and E. coli heterologously expressing the pNL29 GV gene cassette from B. megaterium (OD600=4.46). N=3 for each data point. FIGS. 2B-D show pseudocolored saturation contrast images of a three-compartment phantom containing Microcystis sp. (OD600=1.2), E. coli expressing pNL29 (OD600=5.8), and purified GVs from Halobacteria NRC-1 (OD500, PS=0.32). Saturation was applied at offsets of 58.6 ppm (FIG. 2B), 30.6 ppm (FIG. 2C) and 9.0 ppm (FIG. 2D). FIG. 2E shows a color overlay of FIGS. 2B-D. RF saturation and image averaging parameters are listed in Tables 1 and 2.

FIG. 3A shows a diagram of inducible GV expression in E. coli. Cells (gray ovals) contain the pNL29 gene cluster (red) under control of an IPTG-inducible promoter (blue). GVs (black) are only produced when IPTG is present, according to embodiments of the present disclosure. FIG. 3B shows a graph of saturation contrast generated by E. coli (OD600=0.32) containing IPTG-inducible pNL29 after overnight supplementation with different quantities of IPTG. N=4 for each data point. A straight line was fitted to the data as a visual aide. FIG. 3C shows a saturation contrast image of a three compartment phantom containing E. coli (OD600=1.6) carrying IPTG-inducible pNL29, with and without overnight induction with 50 μM IPTG; or an empty control vector induced with 50 μM IPTG. FIG. 3D shows a diagram of cancer cell labeling strategy. GV (black) are functionalized with anti-Her2 antibodies (orange) via biotin-avidin conjugation (gray, blue). The antibody recognizes the Her2 receptor (red) on SKBR3 cells. FIG. 3E shows a graph of saturation contrast generated by GV-labeled SKBR3 or Jurkat cells. N=3 per data point. FIG. 3F shows a saturation contrast image of three-compartment phantom containing SKBR3 cells labeled with antibody-functionalized GVs, similarly labeled Jurkat cells, and unlabeled SKBR3 cells. RF saturation and image averaging parameters are listed in Tables 1 and 2.

FIG. 4 shows an example of image processing resulting in HyperCEST saturation contrast images, according to embodiments of the present disclosure. Raw ¹²⁹Xe images with off-resonance (FIG. 4A) and on-resonance (FIG. 4B) saturation are shown. FIG. 4C shows a ¹²⁹Xe contrast image (same as FIG. 1F) generated by subtracting FIG. 4B from FIG. 4A and normalizing voxel-by-voxel by FIG. 4A, resulting in a per-voxel saturation. All values in FIG. 4C that fall outside the phantom, defined using the off-resonance image in FIG. 4A, are masked. FIG. 4D shows a ¹H reference image of the phantom shown in FIGS. 4A-D.

FIG. 5 shows broadening of the spectral peak of ¹²⁹Xe by GVs. NMR spectra of ¹²⁹Xe in TMC buffer containing 400 pM intact (red) or collapsed (black) Anabaena flos-aquae GVs. Each spectrum is normalized to its peak amplitude.

FIG. 6 shows saturation spectra of cell culture media. Frequency-dependent saturation spectra for NRC-1, BG11 and LB media, used to culture Halobacteria NRC-1, Microcystis sp. and E. coli, respectively, and present in the samples measured in FIG. 2. N=3 for NRC-1; N=1 for BG11 and LB. Saturation parameters were the same as used in FIG. 2A.

FIG. 7 shows saturation spectra of purified halobacterial GVs. Frequency-dependent saturation spectra for intact and collapsed GVs purified from Halobacteria NRC-1. N=1. Note that the GV saturation peak at ˜14.4 ppm matches that of intact Halobacteria NRC-1 (FIG. 2A) but that aqueous Xe saturation is centered around 195 ppm, as in the GVs shown in FIG. 1C. The spectra are noisier than in other figures because lower pressure and gas flow rate had to be used due to the collapse fragility of Halobacteria NRC-1 GVs. Saturation parameters were the same as used in FIG. 1C.

FIG. 8 shows additional examples of GV transmission electron micrographs. (a) GVs purified from Anabaena flos-aquae imaged with TEM at a lower magnification compared to FIG. 1D. Note the absence of particulate contaminants. A small number of collapsed GVs is visible, which may be present in experimental samples or may result from GV collapse during TEM specimen preparation. (b) TEM of GVs purified from Halobacteria NRC-1. (c) Thin section TEM image of E. coli expressing the pNL29 gene cluster

FIG. 9 shows a hyperpolarized xenon distribution predicted by a pharmacokinetic model. Time course of the concentrations of hyperpolarized xenon in the gas reservoir (Cᵣ, magenta), mouth (Cₘ, blue), lungs (Cᵢ, orange) in panels a and c; pulmonary vein (Cₚ, cyan), cerebral arteries (Cₐ, red) and brain tissue (C_{b}, black) in panels b and d. Panels a-b show the results of the entire 300 second simulation. Panels c-d show the same data, but focused on the first 50 seconds during which C_{b} reaches steady state.

FIG. 10 shows brain concentrations of hyperpolarized xenon and MRI signals in HyperCEST imaging. (a) Predicted concentrations of hyperpolarized xenon (C_{b}) in brain tissue during a HyperCEST imaging sequence in brain regions containing (orange) or devoid of (gray) 400 pM GVs. Black tick marks indicate the timing of image acquisition pulses with flip angle α=20°. The hollow and solid blue bars indicate the timing of off-resonance and on-resonance (at the GV peak) saturation pulses, respectively; saturation is interleaved with image acquisition pulses. (b) Predicted MRI signal acquired from each imaging pulse in brain regions containing (orange) or devoid of (gray) 400 pM GVs. (c) Total signal acquired with and without saturation in brain regions containing (orange) or devoid of (gray) 400 pM GVs

### DETAILED DESCRIPTION

Magnetic resonance imaging contrast agents that include a plurality of gas vesicles configured to associate with a noble gas are provided. Also provided are magnetic resonance imaging methods that include administering to a a subject a contrast agent that includes a plurality of gas vesicles, obtaining a magnetic resonance data of a target site of interest, and analyzing the data to produce a magnetic resonance image of the target site. The subject contrast agents and methods find use in magnetic resonance imaging applications.

Below, the subject MRI contrast agents are described first in greater detail. MRI methods are also disclosed in which the subject MRI contrast agents find use. In addition, multiplex MRI methods and kits that include the subject MRI contrast agents are also described.

### Magnetic Resonance Imaging Contrast Agents

Embodiments of the present disclosure include a magnetic resonance imaging (MRI) contrast agent. The MRI contrast agent may be configured to increase contrast in MRI images of a subject. By an increase in contrast is meant that differences in image intensity between adjacent tissues visualized by MRI are enhanced. In certain embodiments, the MRI contrast agent includes gas vesicles (GVs), such as a plurality of gas vesicles. In certain embodiments, the gas vesicles are genetically encoded gas vesicles. For example, the gas vesicles may be bacterially-derived gas vesicles formed by bacteria, such as photosynthetic bacteria (e.g., cyanobacteria), or the gas vesicles may be archaea-derived gas vesicles formed by archaea, (e.g., halobacteria).

Gas vesicles may be derived from any number of species of bacteria or archaea. For example, gas vesicles may be derived from various prokaryotes, including cyanobacteria such as Microcystis aeruginosa, Aphanizomenon flos aquae and Oscillatoria agardhii; phototropic bacteria such as Amoebobacter, Thiodictyon, Pelodictyon, and Ancalochloris; nonphototropic bacteria, such as Microcyclus aquaticus; Gram-positive bacteria, such as Bacillus megaterium; Gram-negative bacteria, such as Serratia sp.; and archaea, such as Haloferax mediterranei, Methanosarcina barkeri, and Halobacteria salinarium.

In certain embodiments, the gas vesicles are isolated from bacteria or archaea using any convenient method known in the art (See, e.g., Sremac et al., 2008. BMC Biotech 8:9; U.S. Pat. No. 7,022,509; the disclosures of each of which are incorporated herein by reference). In certain instances, gas vesicles are isolated by centrifugally-assisted flotation following cell lysis. In certain instances, aggregation of gas vesicles using flocculating agents (such as polyethylenimines, polyacrylamides, polyamine derivatives, ferric chloride, and alum) enhance the buoyancy of gas vesicles and facilitate isolation of gas vesicles. In certain embodiments, the gas vesicles are substantially spherical in shape. In some instances, the gas vesicles are ellipsoid in shape. Other shapes are also possible depending on the type of bacteria the gas vesicles are derived from. For instance, the gas vesicles may be cylindrical in shape, or may have a center portion that is cylindrical with end portions that are cone shaped, or may be football shaped, and the like.

In certain embodiments, GVs have dimensions that are nanoscale, with exact sizes and shapes varying between genetic hosts. By nanoscale is meant that the average dimensions of the GVs are 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less, or 25 nm or less, or 10 nm or less. For example, the average diameter of the GVs may range from 10 nm to 1000 nm, such as 10 nm to 500 nm, including 10 nm to 250 nm. By “average” is meant the arithmetic mean.

In certain embodiments, the gas vesicle has a vesicle wall. The vesicle wall may be produced by the bacteria the GVs are derived from. For instance, the GVs may have a vesicle wall that is composed of a protein or peptide, such as GvpA. In certain cases, the vesicle wall is a semipermeable vesicle wall. In these instances, the vesicle wall may be permeable to a gas (e.g., air, oxygen, nitrogen, noble gases, such as helium, neon, argon, krypton, xenon), but is substantially impermeable to liquids (e.g., water, saline, buffer, the surrounding fluid media the contrast agent is in during use, etc.). As such, a gas (e.g., a gas from the surrounding media) may substantially freely diffuse in and out of the GVs, whereas liquids are substantially excluded from the interior of the GVs. In these instances, a gas may be said to associate with the GVs. For example, a gas associated with GVs may be in gaseous form inside the GVs and may freely diffuse in and out of the GVs across the gas permeable vesicle wall. In these embodiments, substantially no pressure gradient exists between the inside and outside of GVs, which in some cases may facilitate the stability of the structure of the GVs.

In certain embodiments, the GVs have an interior volume of 5 picoliters (pL) or less, such as 1 pL or less, including 750 femtoliters (fL) or less, or 500 fL or less, or 250 fL or less, or 100 fL or less, or 75 fL or less, or 50 fL or less, or 25 fL or less, or 10 fL or less, or 1 fL or less, or 750 attoliters (aL) or less, or 500 aL or less, or 250 aL or less, or 100 aL or less, or 75 aL or less, or 50 aL or less, or 25 aL or less, or 10 aL or less, or 5 aL or less, or 1 aL or less.

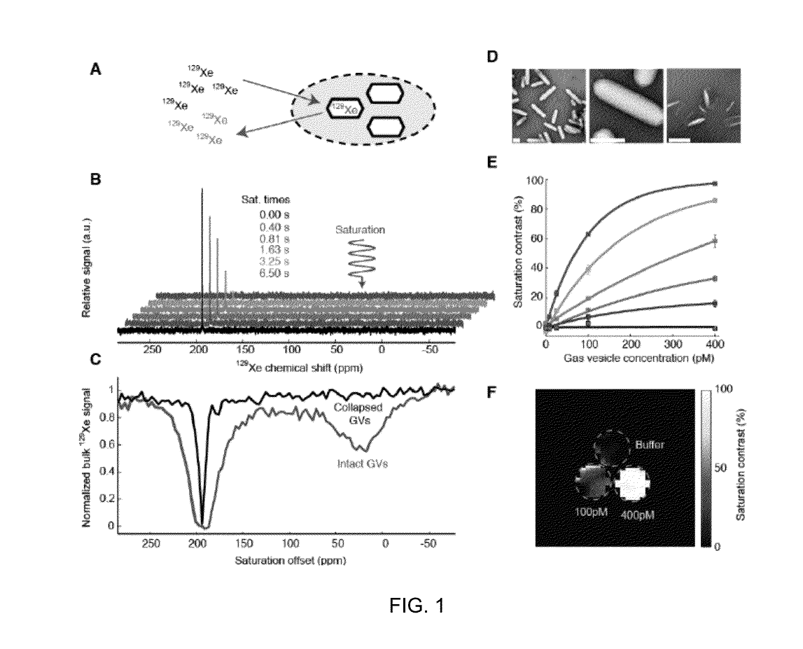

In certain embodiments, the vesicle wall is configured to maintain the shape and size of the GVs under normal usage conditions (e.g., production, isolation, storage, administration to a subject). In certain instances, the vesicle wall has a thickness of 10 nm or less, such as 9 nm or less, including 8 nm or less, or 7 nm or less, or 6 nm or less, or 5 nm or less, or 4 nm or less, or 3 nm or less, or 2 nm or less, or 1 nm or less. For example, FIG. 1A shows a diagram of a gas vesicle (GV) that includes a hollow gas nanocompartment surrounded by a semipermeable protein shell.

In certain embodiments, the GVs include a specific binding moiety attached to a surface of the gas vesicles. The specific binding moiety may be configured to specifically bind to a target site in a subject. For example, the specific binding moiety may include a binding member stably associated with a surface of the gas vesicle. By “stably associated” is meant that a moiety is bound to or otherwise associated with another moiety or structure under standard conditions. In certain instances, the specific binding moiety is stably associated with (e.g., bound to) a surface of the gas vesicle, as described above. Bonds may include covalent bonds and non-covalent interactions, such as, but not limited to, ionic bonds, hydrophobic interactions, hydrogen bonds, van der Waals forces (e.g., London dispersion forces), dipole-dipole interactions, and the like. In certain embodiments, the specific binding moiety may be covalently bound to the gas vesicle. Covalent bonds between the specific binding moiety and the gas vesicle may include covalent bonds that involve reactive groups, such as, but not limited to, N-hydroxysuccinimide (NHS) esters (such as sulfo-NHS esters), imidoesters, aryl azides, diazirines, carbodiimides, maleimides, cyanates, iodoacetamides, and the like.

A binding moiety can be any molecule that specifically binds to a target site of interest, e.g., a protein, peptide, biomacromolecule, cell, tissue, etc. that is being targeted. In some embodiments, the affinity between a binding moiety and its target site to which it specifically binds when they are specifically bound to each other in a binding complex is characterized by a K_{D} (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower K_{D}.

The specific binding moiety can be any molecule that specifically binds to a protein, peptide, biomacromolecule, cell, tissue, etc. that is being targeted (e.g., a protein peptide, biomacromolecule, cell, tissue, etc. at a target site of interest in a subject). Depending on the nature of the target site, the specific binding moiety can be, but is not limited to, an antibody against an epitope of a peptidic analyte, or any recognition molecule, such as a member of a specific binding pair. For example, suitable specific binding pairs include, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a peptide aptamer binding pair; and the like.

In certain embodiments, the specific binding moiety includes an antibody. An antibody as defined here may include fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may also include Fab′, Fv, F(ab′)₂, and or other antibody fragments that retain specific binding to antigen.

In certain embodiments, the antibody may specifically bind to an analyte at the target site of interest. In some cases, the specific binding moiety is stably associated with a gas vesicle, as described above. The gas vesicle-bound specific binding moiety may be configured to specifically bind to an analyte at a target site of interest in a subject. As such, specific binding of the gas vesicle-bound specific binding moiety to the analyte at the target site of interest may indirectly bind the gas vesicle to the target site of interest in the subject. Binding of the gas vesicle to the target site may stably associate the gas vesicle with the target site and thus facilitate detection of the MRI contrast agent containing the gas vesicles and thus facilitate the production of an MRI image of the target site of interest in the subject.

In certain embodiments, the gas vesicle is a collapsible gas vesicle. By collapsible gas vesicle is meant a gas vesicle configured such that the vesicle wall of the gas vesicle may be disrupted by the application of an external force, such as an externally applied pressure. For example, under normal use conditions (e.g., production, isolation, storage, administration to a subject), the gas vesicle may be configured to be substantially stable, such that the physical structure of the gas vesicle is not substantially disrupted. Stated another way, the physical structure of the gas vesicle maintains substantially the same semipermeable integrity under normal use conditions. In some instances, the vesicle wall of the gas vesicle may be disrupted by application of an external force, such as an external pressure. For example, application of an externally applied pressure may disrupt the semipermeable integrity of the vesicle wall such that the vesicle wall is substantially permeable to the surrounding media (e.g., fluids, such as water, saline, buffer, the surrounding fluid media when the contrast agent is in during use, etc.). As such, a collapsed gas vesicle may provide a contrast in an MRI image that is substantially less than the contrast provided by an intact gas vesicle. For instances, a collapsed gas vesicle may provide substantially no contrast enhancement as compared to an intact gas vesicle. In some cases, the externally applied pressure may be generated by applying ultrasound waves (e.g., an ultrasonic pulse) to the gas vesicles sufficient to produce an increase in pressure at the target site of interest where the gas vesicles are located.

In certain embodiments, the contrast agent includes one or more cells containing intracellular gas vesicles. These gas vesicles may be loaded into cells as part of contrast agent preparation, or may be expressed by cells based on gas vesicle encoding genes contained within the cells. Methods of loading gas vesicles into cells may include any convenient method known in the art, including, but not limited to, chemical transfection (calcium phosphate transfection, lipofection, cationic polymer transfection) electroporation, particle bombardment, cell-penetrating peptide-mediated transport, and the like. For example, cell-penetrating peptides (such as TAT, RGD, and Rabies virus-derived peptide) are used to deliver nanoparticles greater than 300 nm into cells (See., e.g., Delehanty et al., 2010. Ther Deliv 1:411; the disclosure of which is incorporated herein by reference).

Genes required for gas vesicle formation may be found in any number of species of naturally occurring bacteria or archaea that produce gas vesicles, as described above. In certain instances, gas vesicle-forming genes may be obtained from these bacteria or archaea. In certain instances, the gas vesicle-forming genes comprise genes that encode proteins that form part of the gas vesicles and/or genes that regulate the expression of genes that encode the gas vesicle proteins. In some instances, the genes required for gas vesicle formation are found in a cluster in the genome of the bacteria or archaea species. The cluster of genes may include a gene encoding for a single highly conserved protein, GvpA, or a closely related homolog thereof, such as GvpB found in Bacillus megaterium. In certain instances, GvpA is the primary protein component of gas vesicles and when assembled forms a hydrophobic inner surface of the gas vesicle and a hydrophilic exterior surface. An exemplary amino acid sequence of GvpA from Anabaena flos-aquae (GID 3683431) is shown below.

(SEQ ID NO: 1)
MAVEKTNSSSSLAEVIDRILDKGIVVDAWVRVSLVGIELLAIEARIVIAS
VETYLKYAEAVGLTQSAAMPA

In certain instances, a 6 kilobase (kb) cluster encoding 11 gas vesicle genes from Bacillus megaterium may be sufficient to confer formation of gas vesicles when heterologously expressed in E. coli. In yet another instance, a 16.6 kb cluster encoding GV genes from Serratia sp. ATCC strain 39006 may be sufficient to confer formation of gas vesicles when heterologously expressed in E. coli. The genes contained in these gas vesicle gene clusters are listed in the table below.

Genes in Gas Vesicle Gene Clusters that are Sufficient to Induce Gas Vesicle Formation when Expressed in E. coli

| Species/Strain | Gene name | GID |
|---|---|---|
| Bacillus | gvpB | 8987738 |
| megaterium | gvpR | 8987737 |
| | gvpN | 8987736 |
| | gvpF | 8987735 |
| | gvpG | 8987734 |
| | gvpL | 8987733 |
| | gvpS | 8987732 |
| | gvpK | 8987731 |
| | gvpJ | 8987730 |
| | gvpT | 8987729 |
| | gvpU | 8987728 |
| | araC | 8987727 |
| Serratia sp. | gvpA1 | 16810365 |
| ATCC strain | gvpC | 16810366 |
| 39006 | gvpN | 16810367 |
| | gvpV | 16810368 |
| | gvpF1 | 16810370 |
| | gvpG | 16810371 |
| | gvpW | 16810372 |
| | gvpA2 | 16810373 |
| | gvpK | 16810374 |
| | gvpX | 16810375 |
| | gvpA3 | 16810376 |
| | gvpY | 16810377 |
| | gvrA | 16810378 |
| | gvpH | 16810379 |
| | gvpZ | 16810380 |
| | gvpF2 | 16810381 |
| | gvpF3 | 16810382 |
| | gvrB | 16810383 |
| | gvrC | 16810384 |

In some embodiments, the cells are of a type that naturally produce gas vesicles. In some embodiments, the cells are bacterial cells heterologously expressing gas vesicles from a plasmid or genome-integrated DNA. “Heterologous,” in the context of two things that are heterologous to one another, refers to two things that do not exist in the same arrangement in nature. In the context of heterologous expression of genes or proteins, genes or proteins are heterologously expressed in a bacterial cell if the genes or proteins are not expressed in a naturally occurring bacterial cell. In some embodiments, the cells are eukaryotic cells, such as mammalian cells, heterologously expressing gas vesicles from a plasmid, viral vector or genome-integrated DNA. In these instances, genes or proteins are heterologously expressed in eukaryotic cells if the genes or proteins are not expressed in naturally occurring eukaryotic cells, such as mammalian cells. Methods for facilitating heterologous expression of prokaryotic genes in mammalian cells are known, including, but not limited to, codon optimization and polycistronic expression (See, e.g., Jinek et al., 2013. Elife 2:e00471; Close et al., 2010. PLoS One 5:e12441; Grohmann et al., 2009. BMC Cancer 9:301; the disclosures of each of which are incorporated herein by reference). In certain instances, mammalian cells heterologously expressing gas vesicles may be autologous or heterologous to the target individual. Such mammalian cells may be, for example, tumor cells, immune cells, stem cells or other cell types.

In certain embodiments, the gas vesicles are encoded genetically in one or more gene vectors, such as a non-viral gene delivery vector, a DNA virus or a RNA virus, and the gene vector or vectors are administered to the subject. Viral gene delivery vectors include, but are not limited to, adenoviruses, adeno-associated viruses, retroviruses and lentiviruses, as well as engineered combinations of natural viral variants, such as pseudotyped, mosaic or chimeric viral vectors. The gene vector may transfect all cells in the area of administration, or may target specific cells based on the characteristics of the vectors. In some embodiments, the vector is designed with promoters such that only a subset of transfected cells, or only under certain intracellular conditions, the gas vesicles are expressed in the target cells.

In certain embodiments, the heterologous expression of gas vesicles from a plasmid, viral vector or genome-integrated DNA is constant, or expression is only under certain times or under certain environmental conditions. In certain embodiments, expression is induced by a specific cue administered to the subject. For example, the specific cue may be a chemical inducer, temperature change, electromagnetic radiation, and the like. For example, expression of gas vesicles may be induced by IPTG, tetracycline, natural and synthetic steroid hormones, and the like.

In certain embodiments, gas vesicle genes are integrated into the genome of a model organism, such that they are expressed in all or a subset of cells in that organism, constantly or at certain times or under certain conditions. In some embodiments, such an organism may be a transgenic mouse, zebrafish, or other species. Methods of integrating genes into the genome of a model organism may include any convenient method of gene targeting known in the art, including but not limited to, viral integration, gamma-ray irradiation, Zinc-finger nuclease-mediated recombination, TALEN-mediated recombination, CRISPR/Cas-mediated recombination, Cre-Lox recombination, FLP-FRT recombination, PhiC31 integrase-mediated recombination, YR-mediated recombination, SR-mediated recombination, and the like.

In certain embodiments, the gas vesicles are configured to be compatible for use in MRI, for example MRI that uses a noble gas (e.g., neon, xenon, such as hyperpolarized xenon, etc.). For example, the spin polarization of ¹²⁹Xe can be increased to a non-equilibrium state (“hyperpolarized”) by optical pumping, increasing its NMR signal by approximately 10⁴. In certain instances, hyperpolarization of ¹²⁹Xe is carried out by spin-exchange with optically pumped alkali metal vapor. In these instances, the electron spin of atomic nuclei of an alkali metal, such as Rb, is initially polarized by irradiating the alkali metal vapor with polarized light.

¹²⁹Xe is a substantially inert and biocompatible element that rapidly distributes into tissues such as the lungs, brain, heart and kidneys after being introduced into a subject in gaseous form, where its polarization decays exponentially with a magnetization lifetime (T1) of 4-6 seconds. Because of its high spin polarization, sub-millimolar local concentrations of ¹²⁹Xe are sufficient for imaging. As a result, in certain embodiments, MRI contrast agents that include xenon may be detectable at low concentrations, e.g., nanomolar, picomolar or lower concentrations.

In certain aspects, hyperpolarized ¹²⁹Xe MRI operates on the basis of chemical exchange saturation transfer (HyperCEST). Because of its high polarizability, xenon's NMR frequency is sensitive to its local chemical environment. HyperCEST contrast agents may produce a distinct chemical shift in ¹²⁹Xe. When radiofrequency (RF) saturation pulses are applied at this frequency, rapid exchange between gas vesicle-contained xenon and dissolved xenon in the surrounding media may result in saturation transfer between these two compartments, reducing the signal in the xenon in the surrounding media. In certain instances, during use, dissolved ¹²⁹Xe in the surrounding media may partition into GVs, where the ¹²⁹Xe may form a gaseous phase with a distinct chemical shift, and may rapidly exchange between GVs and solution, thus allowing GVs to be used as genetically encoded HyperCEST contrast agents (FIG. 1A).

For example, the contents of GVs may be in constant exchange with gas molecules dissolved in surrounding media. In certain instances, the contents of GVs may be in constant exchange with gas molecules dissolved in adjacent tissue. GVs may be permeable to gases ranging in size from hydrogen to perfluorocyclobutane. GVs may include copies of a single highly conserved protein, GvpA, but their formation may use at least 8 genes contained in GV gene clusters. A 6 kilobase (kb) cluster encoding 11 GV genes from Bacillus megaterium may be heterologously expressed in E. coli, conferring the formation of GVs.

### Magnetic Resonance Imaging Methods

Embodiments of the methods are directed to MRI methods. In certain instances, the method includes imaging a target site using a contrast agent, e.g., as described above. As described above, the contrast agent may include a plurality of gas vesicles.

A target site may include may be in vivo or in vitro. As such, a target site may include, for example, any molecule, cell, tissue, body part, body cavity, organ system, whole organisms, collection of any number of organisms, etc., that are of interest. For example, target sites may include a vessel containing a solution comprising a collection of organisms, including, bacteria or archaea. In certain instances, target sites may include a vessel containing a solution comprising cells grown in culture, including, primary mammalian cells, immortalized cell lines, tumor cells, stem cells, and the like. In certain embodiments, target sites of interest include tissue and organs in culture. In certain embodiments, target sites of interest include tissue, organs, or organ systems in a subject, for example, lungs, brain, kidneys, liver, heart, the central nervous system, the peripheral nervous system, the gastrointestinal system, the circulatory system, the immune system, the skeletal system, the sensory system, and the like.

In certain embodiments, administering the contrast agent includes administering the contrast agent produced and prepared outside the subject. In certain embodiments, administering the contrast agent includes administering to the subject one or more gene vectors that contain genes that encode the contrast agent, as described above.

In certain embodiments, the contrast agent is administered to a subject in any pharmaceutically and/or physiologically suitable liquid or buffer known in the art. For example, the contrast agent may be contained in water, physiological saline, balanced salt solutions, buffers, aqueous dextrose, glycerol or the like. In certain embodiments, the contrast agent may be administered with agents that may stabilize and/or enhance delivery of the contrast agent to the target site. For example, the contrast agent may be administered with a detergents, wetting agents, emulsifying agents, dispersing agents or preservatives.

In certain embodiments, the contrast agent is administered locally or systemically. Methods of administering include, but are not limited to, intradermal, intramuscular, intraperitoneal, intravenous, subcutaneous, intranasal, rectal, vaginal, and oral routes. The contrast agent may be administered by any convenient route, for example by infusion or bolus injection, by absorption through epithelial or mucocutaneous linings (e.g., oral mucosa, vaginal, rectal and intestinal mucosa, etc.) and may be administered together with other biologically active agents. In certain embodiments, administering the contrast agent includes injecting the contrast agent into a subject at the target site of interest, such as in a body cavity or lumen. In other embodiments, the administering includes providing the contrast agent in an ingestible formulation that a subject may orally ingest to provide the contrast agent at a desired target site, such as a target site in the digestive tract.

In certain embodiments, a noble gas is administered to a subject or target site thereof. In certain embodiments, the noble gas may be xenon gas. For example, the noble gas may be ¹²⁹Xe gas, such as hyperpolarized ¹²⁹Xe gas. In certain embodiments, the noble gas is administered locally or systemically. The noble gas may be administered to the subject by any conventional means known in the art. For example, the noble gas may be administered to the subject by dissolving the noble gas in the medium in which the subject resides. In certain embodiments, the noble gas may be administered to the subject by inhalation. In yet another embodiment, the noble gas is administered to the subject parenterally in a lipid emulsion. In certain instances, the noble gas is administered to the subject parenterally in a microfoam. In certain instances, the noble gas is administered to the subject by infusion, for example, systemically, or regionally or locally by e.g. intra-arterial, intra-tumoral, intra-venous, or parenteral infusion. In yet other embodiments, the noble gas is administered to the subject by extracorporeal membrane gas exchange.

In certain embodiments, the method includes obtaining an MRI image of the target site in the subject. In some cases, the method includes applying an external magnetic field to the target site in the subject, transmitting a radio frequency (RF) signal from a transmitter to the target site, and receiving MRI data at a receiver. The MRI data may be analyzed using a processor, such as a processor configured to analyze the MRI data and produce an MRI image from the MRI data. In certain embodiments, the MRI data detected by the receiver includes an MRI signal (e.g., a radio frequency MRI signal of the target site of the subject). Additional aspects of MRI systems and methods are found, for example, in U.S. Pat. Nos. 7,307,421, 7,295,008, 7,050,617, 6,556,010, 6,242,916, 4,307,343 the disclosures of each of which are incorporated herein by reference. In certain embodiments, the method includes obtaining a first MRI data (e.g., signal) of the target site, and analyzing the first MRI data (e.g., signal) to produce an MRI image of the target site. The MRI data (e.g., signal) may be obtained using a standard MRI device, or may be obtained using an MRI device configured to specifically detect the contrast agent used. Obtaining the MRI data (e.g., signal) may include detecting the MRI data (e.g., signal) with an MRI detector.

In certain embodiments, MRI data is obtained by applying to a subject a strong static magnetic field, a rapidly switching gradient field for spatial coding, and RF pulses with frequency matched such that the RF pulses trigger magnetic resonance signals from excited atomic nuclei at the target site. For example, an atomic nucleus may produce magnetic resonance signals when the RF pulse has a frequency that matches the resonance frequency (measured in chemical shifts (δ) in parts per million (ppm)) of the atomic nucleus. In such cases, the nucleus absorbs the RF pulse energy to become excited, and releases a magnetic resonance signal when the excited nucleus subsequently relaxes to an unexcited state after characteristic time periods. The magnetic resonance signals are detected by RF receiving antennas and digitized to generate the MRI data. The MRI data is analyzed using any known method of analyzing MRI data. In certain instances, the MRI data is analyzed to reconstruct the MRI image. For example, the MRI image is reconstructed from the MRI data by decoding the spatial information encoded in the MRI data using a linear reconstruction algorithm, such as Fourier transformation.

In certain embodiments, the MRI method includes methods for enhancing contrast in the MRI image. In certain embodiments, methods for enhancing contrast in the MRI image include administering a contrast agent to the target site. For example, the MRI method using a contrast mechanism may be chemical exchange saturation transfer (CEST) MRI. CEST MRI relies on the dependence of the resonance of an atomic nucleus, such as a proton, on the chemical environment of the nucleus, and the ability of the atomic nucleus to exchange at a sufficient rate with another atomic nucleus in a different chemical environment. In other words, the resonance frequency (or chemical shift) of a first exchangeable pool of nuclei in a first chemical environment is offset relative to the resonance frequency of a second exchangeable pool of nuclei in a second chemical environment. In CEST MRI, selective saturation of the first pool of nuclei by applying saturation RF pulses at the resonance frequency of the first pool of nuclei causes a reduction in the signal from the second pool of nuclei between which the first nuclei can exchange. For example, a proton in an amide group (—NH) of a protein and protons in water molecules surrounding the protein have distinct resonance frequencies, and the proton in an amide group in a protein may exchange sufficiently rapidly with protons in the water molecules. Selective saturation of protons in a protein in solution causes progressive saturation of, and thus a decrease in, the MR signal from the protons in the surrounding water due to CEST. As a result, the signal from the protons in the protein are enhanced relative to the surrounding water.

For example, in certain instances, an MRI method includes applying to the target site a saturating radio frequency having a frequency offset relative to the resonance frequency of the noble gas used, such as xenon (e.g., hyperpolarized ¹²⁹Xe), dissolved in the surrounding media. In certain instances, the noble gas is dissolved in adjacent tissue. In certain instances, an MRI method includes applying to the target site a saturating radio frequency having a frequency offset relative to the resonance frequency of the noble gas dissolved in the adjacent tissue. In certain embodiments, the frequency offset is 350 ppm or less, or 300 ppm or less, or 250 ppm or less, or 200 ppm or less, or 150 ppm or less, or 100 ppm or less relative to the resonance frequency of the noble gas dissolved in the surrounding media. For example, the frequency offset may range from 100 ppm to 350 ppm, including 100 ppm to 300 ppm, such as 100 ppm to 250 ppm relative to the resonance frequency of the noble gas dissolved in the surrounding media. In certain embodiments, the frequency offset may range from 100 ppm to 250 ppm relative to the resonance frequency of the noble gas dissolved in the adjacent tissue.

In some instances, the frequency offset is correlated to the type of gas vesicle used, such as the type of bacteria the gas vesicle is derived from. In certain instances, gas vesicles derived from different bacteria have different physical structures (e.g., shape and/or size). In these instances, the gas vesicles derived from different species of bacteria may have different corresponding frequency offsets. As such, gas vesicles derived from different bacteria species may be individually detectable at different frequency offsets, where, for example, a first contrast agent containing a first gas vesicle is detectable at a first frequency offset and a second contrast agent containing a second gas vesicle is detectable at a second frequency offset.

In certain embodiments, the method includes obtaining one or more images of the target site using the resonance frequency of one nucleus, such as ¹H, to obtain images of the anatomy, then obtaining one or more images using the resonance frequency of the hyperpolarized noble gas to obtain an image produced by the presence of the gas vesicles.

In certain embodiments, the method further includes disrupting a vesicle wall of the gas vesicles. For example, the method may include applying a pressure to the gas vesicles sufficient to disrupt a vesicle wall of the gas vesicles. In some cases, the pressure may be provided by applying an ultrasonic pulse to the target site sufficient to disrupt a vesicle wall of the gas vesicles. As described above, the ultrasonic pulse may be sufficient to collapse the gas vesicles such that the gas vesicles do not have a substantial contrast-enhancing effect. In these cases, the method may further include obtaining a second MRI signal of the target site, and analyzing the first MRI signal and the second MRI signal to produce an MRI image of the target site. For example, the first and second MRI signals may be analyzed respectively to produce a first and second MRI images, respectively. In some cases, the first and second MRI signals may be analyzed to produce a composite image of the first and second MRI signals. For instance, the composite image may be a difference image of the first and second MRI signals. As described above, the image obtained after the gas vesicles have been collapsed may not have a substantial contrast-enhancing effect, and as such, a difference image may facilitate an increase in the signal to noise ratio in the resulting composite image.

In some embodiments, the method includes the uniplex analysis of a target site of interest in a subject. By “uniplex analysis” is meant that a contrast agent is administered to a target site and the target site is analyzed to detect an MRI image of the target site. For example, a single type of contrast agent may be administered to the target site and an MRI image of the target site obtained. In some cases, the method includes the uniplex analysis of the target site to determine an MRI image of the target site of interest in the subject.

As described herein, GVs from different species, which have distinct shapes and sizes, may have different chemical shifts. In certain embodiments, GVs from different species operate at unique magnetic resonance frequencies, enabling multiplexed imaging. As such, certain embodiments include the multiplex analysis of two or more contrast agents in a subject. By “multiplex analysis” is meant that the presence two or more distinct contrast agents, in which the two or more contrast agents are different from each other, is determined. For example, contrast agents may be specifically targeted to different target sites in a subject using different specific binding moieties attached to the gas vesicles. In other embodiments, the two or more contrast agents may be different in that they are derived from different species of bacteria. For instance, the contrast agents may be derived from different bacteria, and thus may have a different physical structure, and thus may have different chemical shifts when observed by MRI (or NMR), e.g., hyperCEST imaging. In these instances, a first and second contrast agent may be administered to a target site in a subject. A first MRI signal may be obtained at a first chemical shift, and a second MRI signal may be obtained at a second chemical shift. The first and second MRI signals may be analyzed individually or together to produce individual MRI images of the signals or composite images of two or more of the signals.

In other embodiments, the two or more contrast agents may be a genetically engineered variant of a contrast agent. In these embodiments, at least one protein that is a component of, or contributes to the formation of, a contrast agent, such as gas vesicles, may be altered or deleted by genetic engineering such that the genetically engineered protein confers distinct physical properties (e.g., shape and sizes) and thus confers a distinct chemical shift to gas vesicles compared to gas vesicles that do not result from the genetic engineering.

In some instances, the number of contrast agents is greater than 2, such as 3 or more, 4 or more, 5 or more, etc., up to 10 or more, distinct contrast agents. In certain embodiments, the methods include the multiplex analysis of 2 to 10 distinct contrast agents, such as 3 to 10 distinct contrast agents, including 4 to 10 distinct contrast agents.

### Utility

The subject MRI contrast agents and MRI methods find use in a variety of different applications where producing magnetic resonance image of a subject is desired. In certain embodiments, the subject MRI contrast agents and MRI methods find use in uniplex analysis of a target site in a subject. As described above, the subject MRI contrast agents and MRI methods also find use in the multiplex analysis of a target site in a subject.

Gas vesicle contrast agents thus find use in many molecular imaging applications in cancer, immunology, regenerative medicine and other areas where nanoparticle reporters are desired. In addition, the ability to image GVs inside cells may facilitate GVs use as genetically encoded reporters. GVs are encoded by compact gene clusters (≧6 kb), two of which may be heterologously expressed in Escherichia coli. Bacteria or mammalian cells labeled in this manner may enable non-invasive studies of cellular involvement in processes ranging from infectious disease to organism development. In addition, aggregation-dependent contrast enhancement may facilitate GVs to serve as dynamic molecular sensors for MRI, which may be used to sense concentrations and activities of molecules in vivo.

In addition to their use as MRI molecular reporters, GVs have a variety of anisotropic shapes, hollow interior, gas permeability, optical scattering, buoyancy, abundance of reactive chemical groups, controlled collapse and possibility of genetic engineering, which may facilitate production of GVs with specific properties for the applications described above.

In certain embodiments, the subject MRI contrast agents and MRI methods find use in HyperCEST imaging. In some cases, HyperCEST imaging is ratiometric, making it suitable for imaging even under conditions where the absolute concentration of xenon may be inhomogeneous. In certain instances, the subject MRI contrast agents and MRI methods find use in applications where the use of lower magnetic fields is desired. For instance, ¹²⁹Xe can be polarized without the use of high magnetic fields, allowing molecular imaging and biological assays with comparatively inexpensive low-to-moderate-field MRI magnets.

In certain embodiments, the subject MRI contrast agents and MRI methods find use in research applications. For example, GVs may be used to label and quantify gene expression in bacteria. As such, the subject MRI contrast agents and MRI methods find use in a variety of synthetic biological applications and studies of host-microbe symbiosis, immune defense and tumor growth, etc. In certain cases, GV expression in mammalian cells may facilitate non-invasive imaging of cell expansion, migration and gene expression, for example to facilitate studies of developmental, stem cell, cancer and other biological processes. At ˜6 kb, the size of minimal GV gene clusters may be compatible with the capacity of lentiviral vectors for cell transfection and labeling. Using conjugation techniques, GVs may also find use as exogenous biosensors labeling a wide range of biological targets, for instance, for breast cancer cells. In addition, GVs may be engineered at the genetic level, for example via fusion constructs of GV proteins with other functionalities.

### Kits

Aspects of the present disclosure additionally include kits that include an MRI contrast agent. As described above, the MRI contrast agent includes a plurality of gas vesicles. The gas vesicles may include a specific binding moiety attached to a surface of the gas vesicles and configured to specifically bind to a target site in a subject. In certain embodiments, the kit includes a sterile container containing the MRI contrast agent.

The kits may further include a buffer. For instance, the kit may include a buffer, such as a sample buffer (e.g., saline, phosphate buffered saline, etc.), and the like. The kits may further include additional components, such as but not limited to, sterile wipes, syringes or other administration devices, and the like.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., diskette, CD, DVD, Blu-Ray, computer-readable memory, portable flash drive, etc., on which the information has been recorded or stored. Yet another means that may be present is a website address which may be used via the Internet to access the information at a removed site. Any convenient means may be present in the kits.

As can be appreciated from the disclosure provided above, embodiments of the present invention have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### Example 1

### Materials and Methods

### Cyanobacterial and Halobacterial Cell Culture

Microcystis sp. (CCAP strain 1450/13) and Anabaena flos-aquae (CCAP strain 1403/13F) were purchased from CCAP (Argyll, Scotland, UK) and cultured in sterile BG11 and Gorham's algal media, respectively, at room temperature under fluorescent lighting with an approximately 75% circadian duty cycle. Halobacteria NRC-1 were purchased from Carolina Biological Supply (Burlington, N.C.) and cultured at 37° C. in high-salt medium, under ambient light, according to vendor instructions.

### Gas Vesicle Isolation

Gas vesicles (GVs) were isolated from A. flos-aquae using hypertonic lysis and centrifugally-assisted flotation. Cells were concentrated over a 0.2 μm filter and resuspended in TMC buffer (10 mM Tris-HCl, 2.5 mM MgCl₂, 0.5 mM CaCl₂, pH7.6). A 1:1 volume of 50% sucrose was added rapidly and the cells incubated at room temperature for at least 30 min. The solution was overlaid with a small volume of TMC and centrifuged overnight at 300 rcf. GVs were harvested from the top of the solution. To achieve greater purity, the harvested GVs were resuspended in 10:1 TMC and re-centrifuged and harvested as above; this cycle was repeated 3 times. GVs from Halobacteria NRC-1 were isolated by concentrating the cells through extended floatation, hypotonic lysis with 10:1 TMC, followed by centrifugally assisted floatation as described above. GVs were diluted to experimental concentrations using TMC. To prepare collapsed GVs, GV solutions were loaded into capped plastic syringes and the plunger depressed several times until the solution became translucent.

### Measurement of GV Concentration

The concentration of gas vesicles (GVs) isolated from A. flos-aquae was estimated based on pressure-sensitive OD at 500 nm (OD_{500,PS}) due to intact GV light scattering, measured as the difference in optical density between a solution of intact GVs and the same solution of GVs after popping them through pressure application in a syringe. OD measurements were carried out on the NanoDrop ND-1000 spectrophotometer (Thermo Scientific, Wilmington, Del.) with a path length of 1 mm and scaled to 1 cm. The relationship between OD_{500,PS} and protein concentration (in mg/mL) was determined empirically using a BCA protein assay. Literature-based estimates of the molecular weight of the GVs (93 MDa-121 MDa) were used to calculate the molar concentration. A value of 564.2±94.2 pM/OD_{500,PS} was obtained, which was rounded up to 600 pM/OD_{500,PS}.

Genetic Modification and GV Expression in E. coli

The pNL29 region of the B. megaterium gene cluster containing gvpB through gvpU (Maura Cannon, University of Massachusetts at Amherst) was cloned into the pST39 plasmid for expression under control of the T7 promoter. pNL29-pST39 was transformed into BL21 DE3 E. coli. For tightly regulated IPTG-inducible expression the cells also contained a pLysE plasmid. For saturation spectroscopy and multiplexed imaging, transformed cells without pLysE were grown overnight at 30° C. in selective LB media. For imaging and spectroscopic measurement of gene expression, transformed cells containing pLysE were induced with the indicated concentration of IPTG at OD₆₀₀ of about 0.4 and grown overnight at 30° C. If necessary, prior to experiments cells were concentrated to the specified OD₆₀₀ using a 0.2 μm filter.

### Mammalian Cell Culture and Labeling

SKBR3 cells (ATCC, Manassas, Va.) were cultured in McCoy's 5A medium supplemented with 10% fetal bovine serum (FBS), 100 I.U./ml penicillin and 100 μg/ml streptomycin (pen/step). Before labeling, approximately 5×10⁷ cells were trypsinized and washed twice in phosphate buffered saline (PBS) and once in PBS with 2% bovine serum albumin (BSA). Jurkat T-cells (ATCC) were cultured in RPMI medium supplemented with 10% FBS and pen/strep. Before labeling, approximately 5×10⁷ Jurkat cells were harvested by centrifugation and washed as described for SKBR3 cells. A mouse monoclonal antibody against the human Her2/ERBB2 receptor (clone N12, Thermo Scientific, Fremont, Calif.) was functionalized with streptavidin using the Lightning-Link Streptavidin Conjugation kit following supplier instructions. Purified GVs from A. flos-aquae were biotinylated using EZ-Link Sulfo-NHS-LC-Biotin (Thermo Scientific, Rockford, Ill.) following supplier instructions and purified by floatation. Streptavidin antibodies were conjugated to Biotin-GVs overnight at 4° C. at a 1:4 w/w ratio. To label cells, antibody-conjugated GVs in PBS with 2% BSA were mixed with cells at a GV concentration (based on OD_{500,PS}) of approximately 400 pM. After 1 hour at 4° C., cells were washed twice with PBS and resuspended in 0.6 mL PBS for imaging and spectroscopy.

### HyperCEST NMR

Hyperpolarized xenon was prepared by spin-exchange optical pumping using a homebuilt polarizing apparatus. Briefly, a gas mixture (2% Xe natural abundance, 10% N₂, 88% He) was flowed continuously through the optical pumping cell, which contained approximately 1 g of Rb metal and was heated to produce a vapor. The Rb vapor was irradiated with a linearly-polarized infrared laser (λ=795 nm) to polarize its valence electron, and this electronic polarization was transferred to ¹²⁹Xe upon colliding with Rb via hyperfine coupling. After polarization (˜2%), the gas mixture was delivered to the phantom, an NMR tube (d=5 mm or 10 mm) modified with inlet and outlet ports, through plastic tubing and dissolved in the sample by bubbling through a capillary or set of capillary tubes. Bubbling was controlled using TTL pulses built into the pulse sequence, which in turn controlled pneumatic valves that routed the polarized gas either through the phantom or around it. A 10 second bubble period was followed by a 5 second wait period to allow bubbles to dissipate and the solution to settle. Gas flow rates varied between 0.15 standard liter per minute (SLM) and 0.3 SLM. The entire system, including the phantom, was sealed under a total gas pressure of 1.57 atm to 1.7 atm.

¹²⁹Xe NMR and MRI was performed at 9.4 T on a Varian spectrometer (Palo Alto, Calif.). All experiments were conducted at room temperature, and chemical shifts were referenced to the gaseous ¹²⁹Xe signal. Data were collected using commercial, dual-tuned (1H, broadband) 5 mm and 10 mm probes. For saturation contrast, continuous wave (cw) radiofrequency (RF) pulses with offset frequencies, field strengths and durations specified in Table 1 were applied after the wait period and prior to excitation. Frequency-dependent saturation spectra were obtained by measuring the aqueous ¹²⁹Xe signal as a function of saturation pulse offset, varying from −77.2 ppm to 284.4 ppm in 101 steps. All offsets were relative to ¹²⁹Xe gas.

After data collection, raw FIDs were processed in MATLAB (The MathWorks, Natick, Mass.) by first applying a 10 Hz Lorentzian filter in the time domain before Fourier transform and phase correction. The area of the aqueous ¹²⁹Xe resonance was integrated, and this value was considered for later analyses. To compute saturation contrast, the mean on-resonance signal was subtracted from the mean off-resonance signal (N≧5) under each condition, and the resulting difference was normalized to the mean off-resonance signal. Data and error bars in figures represent the means and standard errors of measurement of biological replicates, with replicate numbers (N) listed in figure captions.

For imaging, a custom phantom was fabricated comprising three 5 mm NMR tubes packed together side-by-side to form a triangle, and fitted with inlet and outlet ports to connect the gas flow from the xenon polarizer. This phantom fit inside of the 10 mm NMR probe. Xenon images were acquired using a fast spin echo imaging sequence, modified to incorporate bubbling and wait periods, as well as a saturation pulse prior to excitation with a 2 ms sinc pulse. Bubbling typically lasted 10 s followed by a 2.5 s wait period, except for experiments with mammalian cells, which used a 7 s bubble and 4 s wait period to minimize foaming. Total gas pressure was maintained between 1.46 atm and 1.57 atm, and the flow rate was either 0.2 SLM or 0.25 SLM.

A train of 8 echoes was used with echo time (TE) of 10 ms, and an overall repetition time (TR) of either 17.58 s (for acquisitions with 7 s bubble and 4 s wait), or 19.08 s (for 10 s bubble and 2.5 s wait). RF saturation was applied immediately after the wait time. Saturation parameters and image averages are listed in Table 2. Signals were acquired with a 12.02 kHz spectral width and 2.66 ms acquisition time. All images were axial without slice-selection, and the k space matrix consisted of 32 points in the readout dimension and 16 phase encoding points. The field of view was 20 mm by 20 mm. The raw matrix was zero-filled by a factor of two in each dimension, manually re-centered in k space, and apodized with a symmetric 2D Gaussian (FWHM=6 cm-1) before 2D Fourier transform to generate images. The root mean square (RMS) noise signal was calculated for a 5 mm by 5 mm square region and images were thresholded starting at 3 times the RMS noise.

Proton images were also acquired with a fast spin echo imaging sequence (TR=1.5 s, TE=16.7 ms, 4 echoes per excitation) after 2 ms sinc excitation, no slice-selection, and 192 points in both readout and phase encode dimensions over a 20 mm by 20 mm field of view. Signals were acquired with a 20.16 kHz spectral width and 9.52 ms acquisition time. The k space matrix was zero-filled once prior to two-dimensional Fourier transform in MATLAB. All proton images are result of 4 averages.

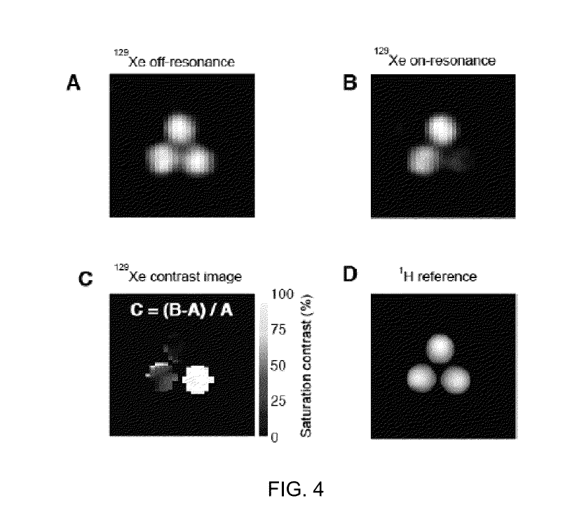

Xenon saturation contrast maps were produced by comparing off-resonance and on-resonance ¹²⁹Xe saturation images (FIG. 4) voxel-by-voxel using custom scripts in MATLAB. The scripts first subtracted the on-resonance saturation image from the off-resonance saturation image to produce a difference image, which was subsequently divided by the off-resonance saturation image thereby normalizing the change in signal. Off-resonance images were used to define regions of interest (ROIs), and the final Xe saturation contrast maps reflect only the contrast within these ROIs. Dashed outlines of the ROIs are overlaid on images as a visual aide.

### Transmission Electron Microscopy (TEM)

TEM images were obtained on a Philips/FEI (Hillsboro, Oreg.) Tecnai 12 microscope operating at 120 kV. GV samples were negatively stained with 2% uranyl acetate and deposited on a carbon-coated formvar grid.

TABLE 1
RF Saturation Parameters Used in HyperCEST Spectrometry

| | | Phantom | | | On- | Off- |
|---|---|---|---|---|---|---|
| | | Diameter | Power | Duration | resonance | resonance |
| FIG. | Specimen(s) | (mm) | (μT, kHz) | (s) | (ppm) | (ppm) |
| 1B | 400 pM GVs | 5 | 33.6, 396 | 0 to 6.5 | 31.2 | n/a |
| 1C | Intact and popped GVs, | 5 | 16.9, 199 | 6.5 | Spectrum | n/a |
| | 400 pM | | | | | |
| 1E | GVs at 0 to 400 pM | 5 | 33.6, 396 | 0 to 6.5 | 31.2 | 356.7 |
| 2A | (i) Microcystis sp. | 10 | (i) 13.5, 159 | 6.5 | Spectrum | n/a |
| | (ii) Halobacteria NRC-1 | | (ii) 12.9, 152 | | | |
| | (iii) E. coli | | (iii) 14.6, 172 | | | |
| 3C | E. coli containing | 10 | 30.1, 354 | 6.5 | 58.6 | 338.3 |
| | pNL29 + quantities of | | | | | |
| | IPTG | | | | | |
| 3F | GV-labeled SKBR3 and | 5 | 23.2, 273 | 6.5 | 31.2 | 356.7 |
| | Jurkat cells | | | | | |

TABLE 2
RF Saturation and Averaging Parameters Used in HyperCEST Imaging

| | | | | On- | Off- | |
|---|---|---|---|---|---|---|
| | | Power | | resonance | resonance | Averages |
| FIG. | Specimen(s) | (μT, kHz) | Duration (s) | (ppm) | (ppm) | per image |
| 1F | 0 pM, 100 pM, 400 pM | 26.9, 317 | 6.5 | 31.2 | 356.7 | 48 |
| | GVs | | | | | |
| 2B | Microcystis sp., | 21.3, 251 | 6.5 | 9.0 | 329.9 | 16 |
| | Halobacteria NRC-1, | | | | | |
| | E. coli pNL29 | | | | | |
| 2C | Microcystis sp., | 21.3, 251 | 6.5 | 30.6 | 329.9 | 16 |
| | Halobacteria NRC-1, | | | | | |
| | E. coli pNL29 | | | | | |
| 2D | Microcystis sp., | 21.3, 251 | 6.5 | 58.6 | 329.9 | 16 |
| | Halobacteria NRC-1, | | | | | |
| | E. coli pNL29 | | | | | |
| 3A | E. coli pNL29 +/− IPTG, | 25.8, 304 | 6.5 | 51.2 | 338.3 | 48 |
| | Control E. coli + IPTG | | | | | |
| 3E | GV-labeled SKBR3 and | 23.1, 273 | 6.5 | 31.2 | 356.7 | 16 |
| | Jurkat cells | | | | | |

### Pharmacokinetic Model of HyperCEST Imaging In Vivo

A previously published pharmacokinetic model of inhaled hyperpolarized xenon was implemented to estimate cerebral tissue concentrations of polarized nuclei and assess the feasibility of HyperCEST imaging in vivo. Model parameters were adjusted to represent rat experimental subjects, and combined with a simulated saturation and imaging pulse sequence to determine its ability to detect the presence of 400 pM GVs in brain tissue. The parameters and variables used in our model implementation are listed in Tables 3 and 4. Equations 1-8 were integrated in MATLAB using the Euler method for 300 seconds using time steps of 10 ms.

$$\begin{matrix}\frac{d {C}_{r}}{d t} = - \frac{{C}_{r}}{{T}_{1 r}} & ( 1 ) \\ \frac{d {C}_{m}}{d t} = - \frac{{C}_{m}}{{T}_{1 m}} + \frac{{f}_{b , i n}}{{V}_{m}} ( {C}_{r} - {C}_{m} ) ( inhalation ) & ( 2 ) \\ \frac{d {C}_{m}}{d t} = - \frac{{C}_{m}}{{T}_{1 m}} + \frac{{f}_{b , out}}{{V}_{m}} ( {C}_{lung} - {C}_{m} ) ( exhalation ) & ( 3 ) \\ \frac{d {C}_{l}}{d t} = - \frac{{C}_{l}}{{T}_{1 l}} + \frac{{f}_{b , i n}}{{V}_{l}} ( {C}_{m} - {C}_{l} ) - \frac{{f}_{p} Θ {C}_{l}}{{V}_{l}} ( inhalation ) & ( 4 ) \\ \frac{d {C}_{l}}{d t} = - \frac{{C}_{l}}{{T}_{1 l}} - \frac{{f}_{p} {ΘC}_{l}}{{V}_{l}} ( exhalation ) & ( 5 ) \\ {C}_{p} = Θ {C}_{l} & ( 6 ) \\ {C}_{a} ( t ) = {C}_{p} ( t - {τ}_{b} ) - exp ( - \frac{{τ}_{b}}{{T}_{1 A}} ) & ( 7 ) \\ \frac{d {C}_{b}}{d t} = {f}_{b} {C}_{a} - {C}_{b} ( \frac{{f}_{b}}{{p}_{b}} + \frac{1}{{T}_{1 b}} + {K}_{sat} ) & ( 8 )\end{matrix}$$

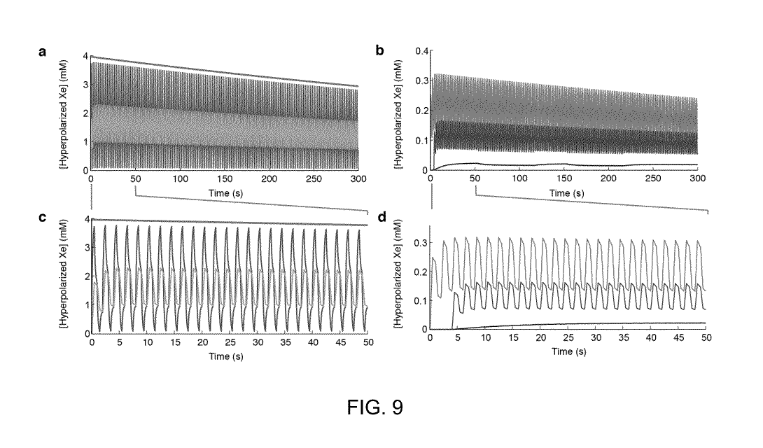

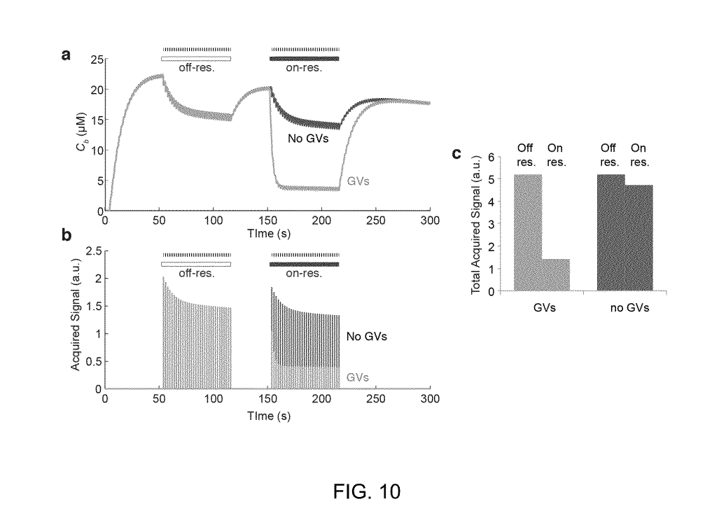

The simulation assumed an initial reservoir concentration of 3.96 mM isotopically enriched hyperpolarized ¹²⁹Xe gas based on a Xe density of 5.15 mg/ml with 10% polarization. The gas was administered through alternating breaths of Xe and O₂. The resulting relative concentrations of ¹²⁹Xe in each compartment are shown in FIG. 9. The peak concentration of hyperpolarized ¹²⁹Xe in brain tissue was predicted to be 22.4 μM (FIG. 9b-d, FIG. 10a). Using the assumptions of Martin et al. (J Magn Reson Imaging 7, 848-854 (1997)), this resulted in a signal-to-noise ratio (SNR) 64-fold lower than that expected for thermally polarized protons based on equation 9, where γ_{Xe} and γ_{H} are the gyromagnetic ratios of ¹²⁹Xe and ¹H (11.77 and 42.577, respectively), C_{H} is the proton concentration (assumed to be 80 M) and the Bₒ field and temperature are taken to be 1.5 T and 310 K.

$$\frac{{SNR}_{Xe}}{{SNR}_{H}} = \frac{{C}_{b} {γ}_{Xe}}{{C}_{H} ( {γ}_{H}^{2} ℏ {B}_{o} / 2 kT )} \qquad ( 9 )$$

In conjunction with the pharmacokinetics, a generalized CEST imaging pulse sequence was simulated, aiming to capture the effect of saturation and acquisition pulses on Xe polarization and estimate the relative acquired signal in the on-resonance and off-resonance conditions. The acquisition sequence contained 32 RF pulses with a flip angle α=20° and a repetition time of 2 seconds. All pulses and signals were assumed to be localized to brain tissues and arteries, e.g., through the use of a surface coil. When C_{b} reached a steady state approximately 50 seconds after the start of the experiment, the acquisition sequence was applied. Each pulse instantaneously reduced Cₐ and C_{b} by a factor of 1-cos(α), and the resulting signal was taken to be proportional to (C_{b}+0.014Cₐ)sin(α), assuming the cerebral vasculature occupies 1.4% of brain volume. After steady state polarization recovered, an imaging sequence was applied again, but was then interleaved with saturation pulses at a GV-selective frequency, starting 2 seconds before the imaging sequence. Saturation pulses in the presence of GVs resulted in a decrease in C_{b} at a rate Kₛₐₜ=0.33 sec⁻¹ in regions containing GVs (equation 8), consistent with the presence of 400 pM GVs and a saturation power comparable to that used in FIG. 1E. No saturation using these frequencies was expected to occur in tissues lacking GVs. The total signal acquired during each imaging sequence was proportional to the sum of the signals produced during component individual pulses.

As shown in FIG. 10b-c, tissues containing GVs were predicted to display saturation contrast (% change between the saturating and non-saturating acquisitions) of 73%. A small change also appeared in the non-GV condition due to the order of image acquisitions and slowly declining Cᵣ, but this could be controlled for by reversing the order of saturating and nonsaturating imaging sequences.

TABLE 3
Pharmacokinetic model parameters

| Symbol | Parameter | Value | |
|---|---|---|---|
| Vₗ | Lung volumeⁱ | 3 | mL |
| Vₘ | Mouth and trachea volume | 1 | mL |
| fₚ | Pulmonary blood flowⁱⁱ | 1.2 | mL sec⁻¹ |
| f_{b} | Cerebral tissue blood flow | 1 L min−1 L⁻¹ | |
| p_{b} | Blood-brain partition coefficient | 1.06 | |
| Θ | Ostwald coefficient | 0.14 | |
| | for Xenon in blood | | |
| τ_{b} | Lung-brain transitⁱⁱⁱ | 4 | sec |
| fᵣ | Respiratory flow rate | 6 | mL sec⁻¹ |
| | (in and out)^{iv} | | |
| T₁ᵣ | T1 time in gas reservoir | 1000 | sec |
| T₁ₘ | T1 time in mouth | 12 | sec |
| T₁ₗ | T1 time in lung | 12 | sec |
| T₁ₐ | T1 time in arterial blood | 6 | sec |
| T_{1b} | T1 time in brain tissue | 15 | sec |
| Kₛₐₜ | HyperCEST on-resonance | 0.33 | sec⁻¹ |
| | saturation rate^{v} | | |

ⁱFor simplicity, we assume that the lung volume and the breath volume are the same and that the lung gets filled with new gas during each inhalation
ⁱⁱBased on a body mass of 300 g
ⁱⁱⁱValue for rats could not be located in the literature; the cited value comes from a study in cats; the rat value is expected to be smaller based on anatomy, which would result in stronger overall xenon signals
^{iv}Based on a breathing rate of 60 breaths min⁻¹ and breath volume of 3 mL (Zhou et al.⁴)
^{v}Assuming local concentration of 400 pM Ana GVs

TABLE 4
Pharmacokinetic model variables

| Symbol | Parameter | Initial value |
|---|---|---|
| Cᵣ | Hyperpolarized xenon concentration | 3.96 mM |
| | in the gas reservoir | |
| Cₘ | Hyperpolarized xenon concentration | 0 |
| | in the mouth and trachea | |
| Cₗ | Hyperpolarized xenon concentration | 0 |
| | in the lungs | |
| Cₚ | Hyperpolarized xenon concentration | 0 |
| | in the pulmonary circulation | |
| Cₐ | Hyperpolarized xenon concentration | 0 |
| | in brain arteries | |
| C_{b} | Hyperpolarized xenon concentration | 0 |
| | in brain tissue | |

### Gas Vesicles Produce HyperCEST Contrast at Picomolar Concentrations

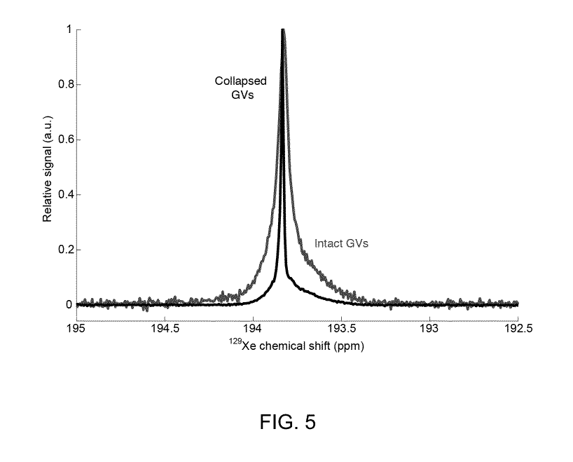

Experiments were performed to test the ability of GVs isolated from Anabaena flos-aquae to produce HyperCEST contrast in aqueous solutions containing hyperpolarized ¹²⁹Xe at 9.4T. At GV concentrations up to 400 pM, no NMR signal other than the main dissolved xenon peak was detectable (FIG. 1B, black). However, RF saturation applied at an offset of 31.2 ppm (relative to gaseous xenon) produced a significant decrease in the dissolved ¹²⁹Xe signal in a saturation-time and power-dependent manner (FIG. 1B). After a 6.5 s exposure to a 33.6 μT (396 kHz) continuous wave (cw) field, the dissolved xenon signal was completely saturated. The dissolved xenon signal was measured as a function of saturation frequency, which showed a unique GV saturation peak at 31.2 ppm (FIG. 1C, red). As a control, GVs were irreversibly collapsed by rapidly increasing pressure above a critical point (FIG. 1D). These collapsed GVs no longer produced saturation contrast (FIG. 1C, black). The presence of intact GVs broadened the direct saturation peak at the chemical shift of aqueous ¹²⁹Xe (FIG. 5), which was characteristic of a chemical exchange interaction.

To determine the molecular sensitivity of GVs as an MRI reporter, experiments were performed to determine HyperCEST measurements across a range of GV concentrations and saturation times (FIG. 1E). With 6.5 s of saturation, 8 pM GVs were sufficient to produce 6.97±0.43% saturation contrast; 400 pM saturated 97.19±1.00% of the xenon signal. With saturation times of 0.4 s and 0.8 s, which were significantly shorter than the in vivo T1 of ¹²⁹Xe, 400 pM GVs produced contrast of 16.17±2.33% and 32.95±1.89%, respectively, and statistically significant contrast was observed at 100 pM. Thus, GV HyperCEST reporters had a molecular sensitivity in the mid-picomolar range. HyperCEST MRI was used to image GVs in a three-compartment phantom containing buffer, 100 pM or 400 pM GVs. Nearly complete saturation was seen in the 400 pM chamber; significant contrast was also present at the lower concentration (FIG. 1F).

Multiplex Imaging of GVs from Different Species

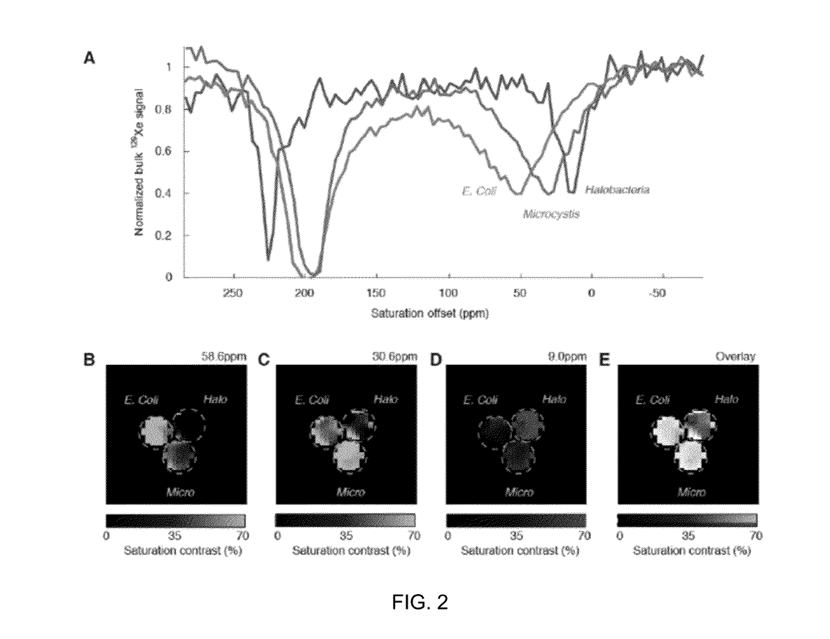

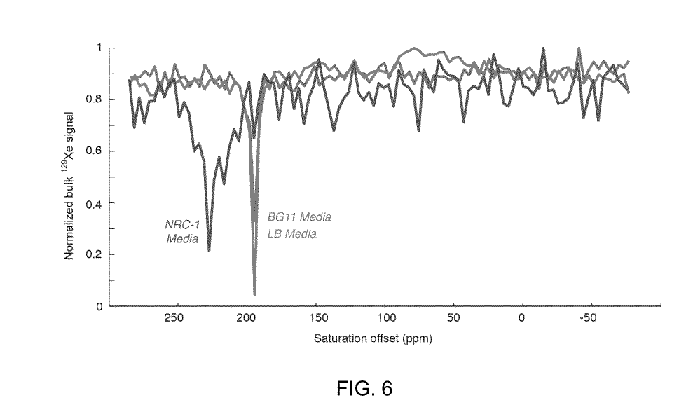

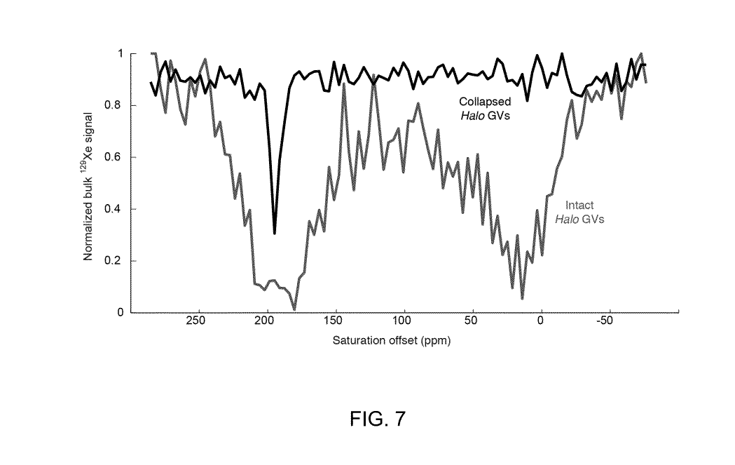

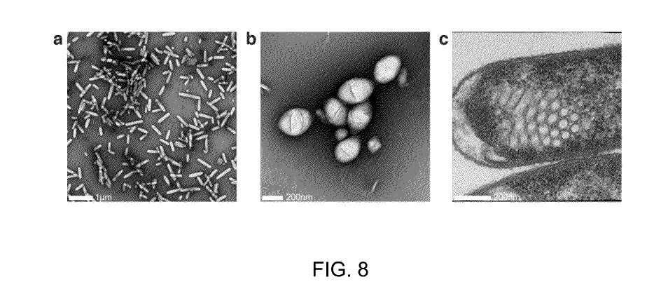

Experiments were performed to determine whether differences in the shape and size of GVs among bacterial species would result in distinct HyperCEST saturation frequencies. Saturation spectra were acquired as a function of frequency from solutions containing intact Halobacteria NRC-1, Microcystis sp., and E. coli transformed with a plasmid containing a minimal GV-forming gene cluster from B. megaterium (FIG. 2A). Each cell type had a unique saturation frequency profile, with maximal saturation at 14.4 ppm, 30.6 ppm and 51.4 ppm for Halobacteria NRC-1, Microcystis sp. and E. coli, respectively. These distinct saturation profiles allowed multiplexed MRI to be performed by applying saturation at three different frequencies (FIG. 2B-E). It should be noted that the downfield-shifted aqueous xenon peak in the halobacterial spectrum at 226 ppm was likely the result of the high salt content of its media (25% NaCl); this shift was also evident in the corresponding media-only saturation spectrum (FIG. 6). In addition, GVs purified from Halobacteria NRC-1 into low-salt buffer (shown in the TEM image in FIG. 8b) produced a broadened aqueous xenon saturation peak centered at the more typical 195 ppm, while the peak attributable to GVs was still centered at approximately 14.4 ppm (FIG. 7). Finally, we note that purified Halobacteria NRC-1 GVs were used in place of intact cells in the MRI images shown in FIG. 2b-e so that the dissolved xenon resonance peak would be consistent across specimens for the purpose of pulse programming.

### Quantitative Imaging of Gene Expression Using Heterologously Expressed GVs

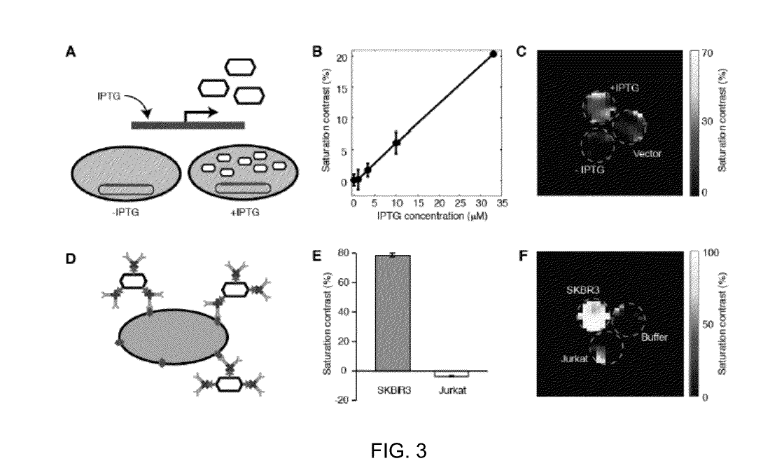

Experiments were performed to determine whether GVs may act as quantitative reporters of gene expression by placing their expression in E. coli under the control of a promoter inducible by isopropyl β-D-1-thiogalactopyranoside (IPTG, FIG. 3A). Overnight induction with IPTG produced enhanced HyperCEST image contrast that was absent from un-induced cells and from induced cells containing a control vector lacking GV genes (FIG. 3B, C). The magnitude of HyperCEST contrast was dependent on the dose of IPTG, confirming the utility of GVs as quantitative reporters of gene expression.

### Non-Invasive Labeling of Breast Cancer Cells Using Biofunctionalized GVs

Experiments were performed to determine the utility of GVs as targeted biosensors. Purified GVs from A. flos-aquae were functionalized with biotin and conjugated them with streptavidin-functionalized antibodies against the Her2 receptor (FIG. 3D). Anti-Her2 GVs were used to label the Her2-expressing breast cancer cell line SKBR3 or control Jurkat cells. A suspension of labeled SKBR3 was distinguishable using HyperCEST imaging from identically treated Jurkat cells (FIG. 3E, F). The targeted breast cancer cells exhibited saturation contrast of 78.53±1.38%.

### Pharmacokinetic Modeling of In Vivo ¹²⁹Xe HyperCEST

Pharmacokinetic modeling was performed to assess the in vivo imaging performance of GV HyperCEST with ¹²⁹Xe-MRI. We adapted published pharmacokinetic models of inhaled hyperpolarized ¹²⁹Xe to include a saturation and imaging pulse sequence. Consistent with previous findings, our model predicted a peak brain tissue concentration of 22 μM hyperpolarized ¹²⁹Xe (FIGS. 9 and 10, assuming the inhalation of isotopically enriched ¹²⁹Xe polarized to 10%). This would result in an MRI signal-to-noise ratio (SNR) 64-fold lower than that of protons but substantially higher than the SNR of ¹⁹F MRI, which is increasingly used for molecular imaging. Importantly, although ¹²⁹Xe magnetization is non-renewable, our model confirmed that the combination of repeated xenon inhalation and low flip angle sequences permits continuous imaging (FIG. 10a-b). Upon the application of an on-resonance saturation pulse, the model predicted a 73% signal decrease in GV-containing regions (compared to an off-resonance control) and minimal change in regions devoid of GVs (FIG. 10b-c). The fact that this detection scheme is ratiometric (i.e. internally normalized by measuring the signal in a given voxel with and without an on-resonance saturation pre-pulse) shows that GV imaging relatively robust to any spatial inhomogeneity in the distribution of xenon in the target tissue. Overall, these modeling results support the feasibility of imaging GV-based HyperCEST reporters in the brain and similarly vascularized organs.

Although the foregoing embodiments have been described in some detail by way of illustration and example for purposes of clarity of understanding, it is readily apparent to those of ordinary skill in the art in light of the teachings of the present disclosure that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A magnetic resonance imaging contrast agent comprising:
a plurality of gas vesicles configured to associate with a noble gas.

2. The contrast agent of claim 1, wherein the noble gas comprises xenon gas.

3. The contrast agent of claim 2, wherein the xenon gas comprises hyperpolarized ¹²⁹Xe gas.

4. The contrast agent of claim 1, wherein the gas vesicles comprise a specific binding moiety attached to a surface of the gas vesicles and configured to specifically bind to a target site in a subject.

5. The contrast agent of claim 4, wherein the specific binding moiety comprises an antibody.

6. The contrast agent of claim 1, wherein the gas vesicles have an average cross-sectional diameter of 40 nm to 250 nm.

7. The contrast agent of claim 1, wherein the gas vesicles comprise a gas permeable protein vesicle wall.

8. The contrast agent of claim 1, wherein the gas vesicles are bacterially-derived gas vesicles.

9. The contrast agent of claim 1, wherein the gas vesicles are archaea-derived gas vesicles.

10. The contrast agent of claim 1, wherein the gas vesicles are heterologously expressed in bacterial or mammalian cells.

11. The contrast agent of claim 1, wherein the gas vesicles are expressed in situ in a subject.

12. A magnetic resonance imaging method comprising:
administering to a subject a noble gas and a contrast agent comprising a plurality of gas vesicles;
obtaining a magnetic resonance data of a target site; and
analyzing the data to produce a magnetic resonance image of the target site.

13. The method of claim 12, further comprising applying a saturating radio frequency to the target site.

14. The method of claim 13, wherein the saturating radio frequency has a frequency offset relative to the resonance frequency of the noble gas dissolved in adjacent tissue.

15. The method of claim 14, wherein the frequency offset has a chemical shift from 100 to 250 parts per million relative to the resonance frequency of the noble gas dissolved in adjacent tissue.

16. The method of claim 13, wherein the obtaining the magnetic resonance data comprises detecting a first magnetic resonance data when the saturating radio frequency is applied.

17. The method of claim 16, further comprising detecting a second magnetic resonance data when the saturating radio frequency is not applied.

18. The method of claim 17, wherein the analyzing comprises analyzing the first and second magnetic resonance data to produce the magnetic resonance image.

19. A multiplex magnetic resonance imaging method comprising:
administering to a subject a noble gas and two or more contrast agents each comprising a plurality of gas vesicles;
applying to a target site a first saturating radio frequency having a first frequency offset relative to the resonance frequency of the noble gas dissolved in adjacent tissue;
obtaining a first magnetic resonance data of the target site;
applying to the target site a second saturating radio frequency having a second frequency offset relative to the resonance frequency of the noble gas dissolved in a surrounding tissue;
obtaining a second magnetic resonance data of the target site; and
analyzing the first and second magnetic resonance data to produce a magnetic resonance image of the target site.

20. The method of claim 19, wherein the analyzing comprises producing a composite image of the first and second magnetic resonance data.

21. The method of claim 19, wherein the first frequency offset is correlated to a first contrast agent and the second frequency offset is correlated to a second contrast agent.

22. The method of claim 19, wherein the gas vesicles are bacterially-derived gas vesicles.

23. The method of claim 19, wherein the gas vesicles are archaea-derived gas vesicles.
